(19) United States
(12) Patent Application Publication
(10) Pub. No.: US 2010/0211147 A1
(43) Pub. Date: Aug. 19, 2010

(54) ELECTRICALLY CONDUCTING MATERIALS, LEADS, AND CABLES FOR STIMULATION ELECTRODES

(75) Inventors: Herwig Schiefer, Frankfurt (DE); Christoph Vogt, Saint Paul, MN (US); Lars Gusseck, Yverdon-les-Bains (CH); Matthias Frericks, Hanau (DE); Heiko Specht, Hanau (DE); Jens Troetzschel, Bruchkoebel (DE)

Correspondence Address: DICKE, BILLIG & CZAJA, FIFTH STREET TOWERS, 100 SOUTH FIFTH STREET, SUITE 2250, MINNEAPOLIS, MN, 55402, US

(73) Assignee: W. C. HERAEUS GMBH, Hanau (DE)

(21) Appl. No.: 12/708,243

(22) Filed: Feb. 18, 2010

(30) Foreign Application Priority Data
Feb. 19, 2009 (DE) 10 2009 009 557.8

(51) Int. Cl. A61N 1/05; H01B 5/08

(52) U.S. Cl. 607/116; 607/119; 174/128.1

(57) ABSTRACT

One aspect is a stranded wire containing numerous coils. The stranded wire is configured as an electrical connection between an electrical stimulation device that is connected to the proximal end of the stranded wire, and an electrode connected to the distal end of the stranded wire. At least one coil includes a tantalum or niobium-based metal.

### CROSS-REFERENCE TO RELATED APPLICATIONS

This Utility Patent Application claims priority to German Patent Application No. DE 10 2009 009 557.8, filed on Feb. 19, 2009, which is incorporated herein by reference. This Patent Application is also related to Utility Patent Application filed on even date herewith, entitled “COILED RIBBON AS CONDUCTOR FOR STIMULATION ELECTRODES” having Attorney Docket No. W683.101.101/P11042 US.

### BACKGROUND

One aspect refers to coiled wire and ribbon assemblies, and cables as electrical leads for stimulation electrodes.

Electrical leads for stimulation electrodes for the purpose of cardial stimulation or neurostimulation must maintain their function for the entire time that the implant remains in the body. They must be biocompatible, electrically conducting, and ductile but also exhibit great mechanical tensile strength.

Due to the high mechanical stresses which affect the electrical leads of the stimulation electrodes while in the human body, for example, through continually recurring cardiac contractions, the materials for said electrical leads are exposed to constant alternating flexural stress.

Usually, such cables are coated with a silicone or PU layer. Clad materials with a core of pure, well conducting material, such as silver, gold, copper, or aluminum have become prevalent as conductors, whereby silver is most commonly used. As coating materials, cobalt alloys have prevailed, especially MP35N® (essentially Co—Cr—Ni—Mo, standardized in accordance with ASTM F562). For some applications, where a somewhat lower conductivity suffices, cables and coiled wires made of MP35N® full material are utilized.

U.S. Pat. No. 6,191,365 discloses medical devices with numerous coiled and drawn wires.

U.S. Pat. No. 6,278,057 discloses coiled and drawn wires, one of which, at least, consists of a nickel-titanium alloy. Additional wires contain stainless steel, platinum, gold, silver, copper, aluminum, nickel, chromium, platinum, iridium, or tungsten.

EP 0 929 343 describes an electrode cable for electrical stimulation made of coiled wires with a conductive core material of silver, gold, aluminum, or copper, a material with high resistance, such as the cobalt alloy MP35N®, and a silicone or PU-based insulating material. The cable has a diameter of 200 μm.

EP 1 718 363 discloses a twisted and bundled wire configuration, which is drawn to the required diameter and coated with insulating material.

U.S. Pat. No. 7,138,582 discloses metallic conductor bundles (so-called leads) made of modified MP35N®, a cobalt alloy with decreased titanium nitrite inclusions.

U.S. Patent Application No. 2006/283621 discloses bundles of aluminum wires with a PVD coating of tin or zinc.

U.S. Pat. No. 5,796,044 discloses various configurations of wires for a so-called biomedical lead, consisting of a conductive wire and an insulating mantle.

U.S. Pat. No. 5,796,044 discloses a conductor for cardiac pacemakers with a Teflon/silicone jacket.

EP 1 827 575 discloses a configuration, in which a wire core consists of silver and is surrounded by an insulating metal wire made of nickel titanium, MP35N®, titanium, or titanium alloy.

U.S. Pat. No. 7,020,947 discloses a metal wire with filaments for biomedical application. Thereto, holes are drilled into a cylinder, conductive material inserted therein, and a wire drawn therefrom. A biocompatible layer forms the outer skin.

WO 2008/054259 uses an electrically conductive ribbon, which is feather-like coiled.

The term “implantable stimulation lead” is supposed to describe the meaning of the word “lead,” which is designated as a technical term for such electrical connections between distal and proximal ends of a cable. A lead is a medical electrical cable with proximal and distal ends for the electrical connection between a device for stimulation and an electrode connected to said device. Leads are usually designed for a plug-in connection with the device. The electrical conductor inside the lead is a stranded wire and/or at least one coiled wire and outwardly electrically insulated, for example, as cable or a coil, which is surrounded by an insulation sleeve.

A stranded wire consists of a multitude of wires twisted around each other and is, therefore, a flexible conductor. The large number of wires provides a redundancy with regard to the function of the electrical conductivity in the event of a wire breakage.

A clad material for the medical electrical lead consists of a core made of a material with high electrical conductivity, for example, Ag, Au, Pt, Cu, Al, and a biocompatible coating with good mechanical properties, for example, MP35N®.

In a cable for the medical electrical lead, a stranded wire or a coiled wire is embedded in an electrical plastic insulation (for example, polyurethane, ETFE, PTFE, or silicone).

If an electrical lead to a stimulation electrode, the cable or coiled wire of which exhibits an MP35N® outer surface, is coated with polyurethane for the purpose of electrical insulation, the elements Cr, Co, and Mo, contained in MP35N®, cause an oxidative degradation of the surrounding PU layer. This was described in EP 0 329 112 correspondingly. Therein, the degradation was decreased through the coating of the metallic conductor with an inert coating of Pt, for example.

With decreasing wire diameter, for example, during the processing of clad materials into cables, the wall thickness of the MP35N® jacket becomes very thin, that is, it drops below a thickness of approximately 5-10 μm. Contaminants in the form of inclusions, frequently occurring in pyro-metallurgical material, can act as trigger for breakage. Particularly through permanent, constantly changing stress, as it occurs through body or organ movement, cracks in the wire can form which lead to failure of the wire and, consequently, the cable.

The described clad materials are manufactured into wire through core boring of a cylindrical full material, subsequent tube manufacturing, insertion of the core material into the tube and concluding drawing of the compound. Contaminants in the form of metal residues and particles in the jacket, in the core, or on the boundary between jacket and core remain in the material during wire manufacture and can lead to significant problems during the drawing process itself as well as the subsequent application. Due to permanent, consistent stress, cracks can form in the core as well as the jacket material.

Furthermore, an aging process of the material occurs with the mostly used MP35N®. Through a phase transformation of the crystalline structure at room temperature, material embrittlement occurs, that is, an increase in strength occurs, however, the elasticity of the material decreases simultaneously. This may result in unforeseeable failure of the material.

For these and other reasons there is a need for the present invention.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

In the following, the hereto is illustrated with the use of examples with reference to the drawings.

FIG. 1 illustrates a single coil.

FIG. 2 illustrates a cable with multi-coil.

FIG. 3 illustrates a cable with a 1×7 strand.

FIG. 4 illustrates a 1×19 strand.

FIG. 5 illustrates a 7×7 strand.

FIG. 6 illustrates a clad material coil.

FIG. 7 illustrates a multi-coil from clad material.

FIG. 8 illustrates a 1×7 strand from clad material.

FIG. 9 illustrates a multi-coil.

FIG. 10 illustrates a 1×7 strand with Ag wire.

FIG. 11 illustrates a 7×7 strand, consisting of 7 1×7 strands.

FIG. 12 illustrates the manufacture of a laminate through roll cladding.

FIG. 13 illustrates a ribbon formed into a coil.

FIG. 14 illustrates a plastic-wrapped ribbon.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

One aspect herein consists of further improving the reliability, for example, the corrosion stability, the workability, and the mechanical stability of medical cables for stimulation electrodes.

In one embodiment, wires made of a Ta or Nb-based metal with high purity and high electrical conductivity are doped or alloyed, subsequently exhibiting a substantially higher mechanical strength while maintaining good electrical conductivity.

In one embodiment, wires made of Ta or Nb-based alloys with excellent mechanical strength, for example, alloys which contain at least one element of the group tantalum, niobium, tungsten, molybdenum, and zirconium, are used.

In one embodiment, wires made of a metal with high purity and good electrical conductivity are embedded, for example, sheathed, wrapped, or laminated, in materials on Ta or Nb basis with excellent strength.

According to one embodiment, an implantable stimulation lead contains a stranded wire, a coil, a metallic composite or a cable as described below. Proven implantable stimulation leads contain, on the proximal end, a plug serving as connection to a pacemaker, an implantable defibrillator, a peripheral muscle stimulator, or a neurostimulator.

Coils on the basis of tantalum or niobium, according to one embodiment, exhibit excellent mechanical properties with regard to the required flexibility for a connection between an electrical stimulation device, for example, a pacemaker, defibrillator, etc., connected at the proximal end, and an electrode connected at the distal end of the coil. However, with regard to materials with comparable good mechanical properties, coils on the basis of tantalum or niobium exhibit a significantly higher conductivity. Consequently, electrical conductivity of the connection can be improved and precious metal saved. According to one embodiment, the possibility of providing electrical conductors on the basis of tantalum or niobium is presented. Furthermore, good electrical conductors, for example, made from silver or gold, can be embedded between bodies on the basis of tantalum or niobium, for example, through wrapping with wires on the basis of tantalum or niobium to a stranded wire or as a compound, for example, as clad material or laminate.

Stranded wires with a multitude of coils on the basis of tantalum or niobium are especially reliable, for example, coiled stranded wires. If applicable, additional good conductors, for example, silver wires, are embedded in tantalum or niobium-based coils.

According to one embodiment, a tantalum or niobium-based metal with a strength greater than 1000 MPa, for example greater than 1200 MPa, replaces the application of MP35N®. With a specific electric resistance below 100 μΩcm, and in one example below 50 μΩcm, and in another below 20 μΩcm, the tantalum or niobium-based metal already contributes significantly to the improvement of the electrical conductivity, or, accordingly, saves precious metal, for example, silver or gold. At a specific electric resistance below 20 μΩcm, the tantalum or niobium-based metal is a good conductor with distinctly better mechanical properties.

In one embodiment, the tantalum or niobium-based metal is doped with an element from the group P, B, O, C, N, Si, F, Zr, Y, La, Ce, Pr, Nd, Sm, Eu, Gd, Tb, Dy, Ho, Er, Tm, Yb, and Lu, and exhibits a specific electric resistance <20 μΩcm. Such doped niobium or tantalum is called fine-grained stabilized tantalum or niobium. IN one embodiment, the tantalum or niobium-based metal is a gradient material, surface-treated with one of the above elements. At a specific electric resistance <20 μΩcm, the doped niobium or tantalum can be used as good electrical conductor, for example as a replacement for silver or gold. Thereby, niobium and tantalum are more biocompatible than silver.

In one embodiment, the tantalum or niobium-based metal is alloyed with one other element from the group niobium, tantalum, tungsten, zirconium, and molybdenum, for example, 0.1-70% w/w Nb, 0.1-30% w/w of at least one element from the group W, Zr, Mo, and less than 5% from at least one of the elements from the group hafnium, rhenium, lanthanides, cerium, and the rest Ta. These alloys exhibit particularly good mechanical properties and, compared to MP35N® (with a specific electric resistance of 103 μΩcm) add significantly to the electrical conductivity.

In one embodiment, wires or metallic conductors with high electrical conductivity, for example, extra-fine wires or conductors made of copper, silver, gold, or aluminum, are surrounded by a metal with greater mechanical strength, for example, by a jacket or a wrapping with extra-fine wires. In one embodiment, the metal with the high electrical conductivity has a specific electric resistance of less than 12 μΩcm.

In one embodiment the conductor of a stranded wire consists of a metal, for example, silver, with a better conductivity than the tantalum or niobium-based metal and is surrounded by a body made of a tantalum or niobium-based metal, for example, a jacket or a coil or several coils.

Whether the conductor made of the metal with the higher electrical conductivity is surrounded by doped tantalum or niobium or by a body made from a niobium or tantalum alloy depends on the requirements regarding diameter, electrical conductivity, and mechanical resilience.

In one embodiment, a conductor made of doped tantalum or niobium-based metal is surrounded by a jacket or a coil or several coils made of tantalum or niobium-based alloy. A coil or helix serves as electrical connection between an electrode and an electrical stimulation device, such as a pacemaker, defibrillator, etc., that is, and electrically connect the stimulation device at the proximal end of the coil with the electrode at the distal end of the coil.

In one embodiment, such a coil is made of a tantalum or niobium-based metal. In one embodiment, the tantalum or niobium-based metal is doped with an element from the group P, B, O, C, N, Si, F, Y, La, Ce, Pr, Nd, Sm, Eu, Gd, Tb, Dy, Ho, Er, Tm, Yb, and Lu in such a way that it exhibits a specific electric resistance <20 μΩcm. In one embodiment, the tantalum or niobium-based metal is a gradient material surface-treated with one of the elements listed above. In one embodiment, the tantalum or niobium-based metal is alloyed with at least one other element from the group niobium, tantalum, tungsten, zirconium, and molybdenum in order to achieve a strength greater than 1200 MPa.

Tantalum or niobium-based metal coils are applicable as an alternative to stranded wires. Tantalum or niobium-based metal coils are also suitable as strands in a stranded wire. The material properties of the tantalum or niobium-based materials, for example, mechanical strength and electrical conductivity, correspond with the doped metals, alloys, gradient materials, and clad materials described elsewhere herein.

Furthermore, the technique described for cables and stranded wires can also be realized in the form of coils. Respective clad materials with a jacket made of tantalum or niobium-based metal are a composite material, whereby the jacket corresponds with the coils, which surround a conductor with better electrical conductive metal. In this context, a metallic composite material is also part of one embodiment herein, whereby a conductor is surrounded, for example, embedded, and the embedding, for example, a jacket, consists of a tantalum or niobium-based metal, whereby the compound is suited to serve as an electrical connection between an electrical stimulation device, such as a pacemaker, defibrillator, etc., which is connected to the proximal end of the metallic composite material, and an electrode connected to the distal end of the metallic composite material.

In one embodiment, the tantalum or niobium-based metal of the composite material exhibits a strength of more than 1000 MPa and a specific electric resistance <200 μΩcm. Doping of a tantalum or niobium-based metal with an element from the group P, B, O, C, N, Si, F, Zr, Y, La, Ce, Pr, Nd, Sm, Eu, Gd, Tb, Dy, Ho, Er, Tm, Yb, and Lu facilitates a specific electric resistance <20 μΩcm.

This also facilitates a compound, for example clad material, in which an electrical conductor made of tantalum or niobium-based metal is embedded.

With a tantalum or niobium-based metal, which is alloyed with another element from the group niobium, tantalum, tungsten, zirconium, and molybdenum, a strength greater than 1000 MPa can be achieved, for example, a strength greater than 1200 MPa.

A lead for stimulation electrodes, according to one embodiment, contains within its electrical insulation at least one metal coil,
which in the case of a coiled wire consists of a tantalum or niobium-based metal;
which in the case of a metal compound, for example, clad material, contains at least one tantalum or niobium-based metal, for example, with a greater mechanical strength than the other metals contained therein;
which in the case of one or more wrapped or coiled coils is the strand of a stranded wire, which contains at least one tantalum or niobium-based metal with greater mechanical strength than the other metals contained therein.

In the simplest case scenario, the electrical lead for stimulation electrodes consists of a coiled electrical conductor with insulation. Such an electrical lead for stimulation electrodes may also contain a single coiled conductor as well as at least one stranded wire.

In a cable for stimulation electrodes, whereby within the insulation a metal with good electrical conductivity is embedded in a metal with great mechanical strength, the metal with great mechanical strength, according to one embodiment, is an alloy on the basis of the elements tantalum, niobium, tungsten, and zirconium. A gradient material has proven successful, whereby only the surface was treated through doping and which, therefore, exhibits increased strength.

The metal with good conductivity is, in one embodiment, an element of high purity, for example, silver, copper, aluminum, or gold.

Compared to MP35N®, precious metal can be saved, according to one embodiment, because the alloys, according to one embodiment, exhibit improved conductivity when compared to MP35N®, therefore, less precious metal is needed for the required conductivity of the clad material.

Furthermore, according to one embodiment, the applicable alloys for embedding of good conductors are already X-ray opaque due to refraction metals, therefore, efforts for a visualization of electrical leads for stimulation electrodes in the X-ray image are no longer required, according to one embodiment.

In one embodiment, good electrical conductors, for example, made from silver, gold, copper, or aluminum, are laminated with a material of high mechanical strength. Suitable materials with excellent mechanical strength are alloys based on tantalum or niobium, which contain at least one more element from the group Ta, Nb, W, Mo, Zr. Said metal laminate is formed into a coil and sheathed with electrically insulating plastic or initially sheathed with electrically insulating plastic and then formed into a coil. The coil, for example, the one initially sheathed with plastic and subsequently coiled, is as coiled ribbon, in one embodiment, sheathed a second time with plastic. In one embodiment, a cavity remains in the center of the coiled ribbon, thereby providing for a hose-like cable.

In one embodiment, cables for permanently implanted medical applications are provided as cables with an outer insulation, inner wires with high electrical conductivity, and wires coiled around the inner wires with great mechanical strength. A wire or wire bundle with exceptionally good electrical conductivity consists, for example, of copper, silver, gold, or aluminum. The wire or wire bundle with good electrical conductivity is wrapped with twisted wires, which exhibit especially good mechanical strength and high biocompatibility. The outer insulation consists of a biocompatible plastic, for example on the basis of an organic polymer which possibly contains fillers. Silicone and PU-based plastics as well as fillers for increasing the X-ray opacity, or for the modification of the electrical conductivity at high frequencies, have proven successful.

In one embodiment, instead of clad materials, wires with great tensile strength are, therefore, twisted with wires with high electrical conductivity. Compared to clad materials, the twisted wires are more kink-proof and, therefore, exhibit improved long-term stability. Proven materials with good electrical conductivity for the inner wires of the strand bundles are silver, gold, copper, or aluminum. For the fine wires for wrapping of the wires with good conductivity, alloys are well-suited, for example, cobalt-free alternative materials to MP35N® standards:

TaNbW, TaNbZr, TaNbWZr, fine-grained stabilized Ta, fine-grained stabilized Nb, P-doped Nb, P-doped Ta, NbZr1, TaW7.5, TaW10, P-doped TaNbW, B-doped TaNbW, O-doped TaNbW, Zr-doped TaNbW, La-doped Ta, gradient materials (for example, externally O-doped NbZr, or externally O-doped TaNbW).

This also allows for the manufacture of clad materials with jackets made of the aforementioned materials, for example, with a core made of conductive material (for example, an Ag core).

With such a clad material for stimulation electrodes with a core made from especially conductive metal, for example with a specific electric resistance of less than 12 μΩcm, such as Ag, Cu, Al, or Au, and a jacket made from tantalum or niobium-based metal, a strength of more than 1000 MPa and a specific electric resistance <100 μΩcm is achieved, according to one embodiment. In one embodiment, the jacket consists of fine-grained stabilized tantalum or niobium. In one embodiment, the jacket is a niobium or tantalum alloy which is alloyed with another element from the group tantalum, niobium, tungsten, zirconium, and molybdenum.

In one embodiment, the wires are coiled or twisted (stranded), for example, to 1×7, 7×7, 1×3, 7×19, 19×7, or 1×19 constructions.

Electrical leads, consisting of extra-fine wires with diameters of less than 200 μm, for example, between 10 and 100 μm, have proven successful. In one embodiment, the diameter of extra-fine wires measures 15 to 50 μm. For ribbons, the profile corresponds with the previously described extra-fine wires.

For achieving high mechanical strength and low electric resistance, electrical leads for stimulation electrodes, which contain a metal with good electrical conductivity and a metal with great mechanical strength embedded in a plastic insulation, for example, made of silicone or PU, exhibit, according to one embodiment, an alloy on the basis of the elements tantalum or niobium, for example, from the system tantalum, niobium, tungsten, molybdenum, and zirconium, as the metal with high mechanical strength.

According to one embodiment, a stimulation electrode is provided with an electrically conductive lead for the conduction of electrical signals to electrode poles at the distal end of the stimulation electrode, the lead of which exhibits a strength of more than 1000 MPa and a specific electric resistance of less than 100 μΩcm, for example, less than 20 μΩcm. According to one embodiment, the electrical lead thereto contains a tantalum or niobium-based metal ribbon.

In one embodiment,
the electrical lead consists of fine-grained stabilized tantalum or niobium and exhibits a specific electric resistance <20 μΩcm, for example, less than 17 μΩcm;
the electrical lead consists of a tantalum or niobium alloy, which contains at least one other element from the group niobium, tantalum, tungsten, zirconium, and molybdenum;
the electrical lead made of tantalum or niobium is a gradient material which is wire surface-doped with at least one element from the group P, B, O, C, N, Si, F, Zr, Y, La, Ce, Pr, Nd, Sm, Eu, Gd, Tb, Dy, Ho, Er, Tm, Yb, and Lu;
the electrical lead exhibits a metal, such as Ag, Au, Cu, or Al, with better conductivity than the tantalum or niobium-based metal, whereby the metal with better conductivity is surrounded by the tantalum or niobium-based metal, for example, embedded or sheathed.

In one embodiment, the metal with the higher electrical conductivity is embedded in fine-grained stabilized tantalum or niobium or a niobium or tantalum alloy, which contains at least one other element from the group niobium, tantalum, tungsten, zirconium, and molybdenum.

During fine-grain stabilization, a fine-grained structure is produced through doping with impurity atoms, which leads to increased strength of the material. In these materials, said impurities stabilize the grains in such a way that unwanted grain growth is suppressed even at prolonged temperature manipulations. Aside from their mechanical strength, fine-grained stabilized tantalum or niobium exhibit good electrical conductivity and can, therefore, further contribute to the conductivity of the entire lead, when compared to materials used heretofore for increasing strength.

For doping, at least one element is used, for example, from the following elements: P, B, O, C, N, Si, F, Zr, Y, La, Ce, Pr, Nd, Sm, Eu, Gd, Tb, Dy, Ho, Er, Tm, Yb, and Lu. Proven dopings are in the range between 50-1,000 ppm per element, for example, in the range between 300-500 ppm, with the sum of all doping elements in the range between 300-10,000 ppm, for example, in the range between 500-5,000 ppm.

According to one embodiment, a stranded wire, a coil, a metallic composite, a cable, or an implantable stimulation lead is used for a cardiac pacemaker, implantable defibrillator, neurostimulator, or peripheral muscle stimulator.

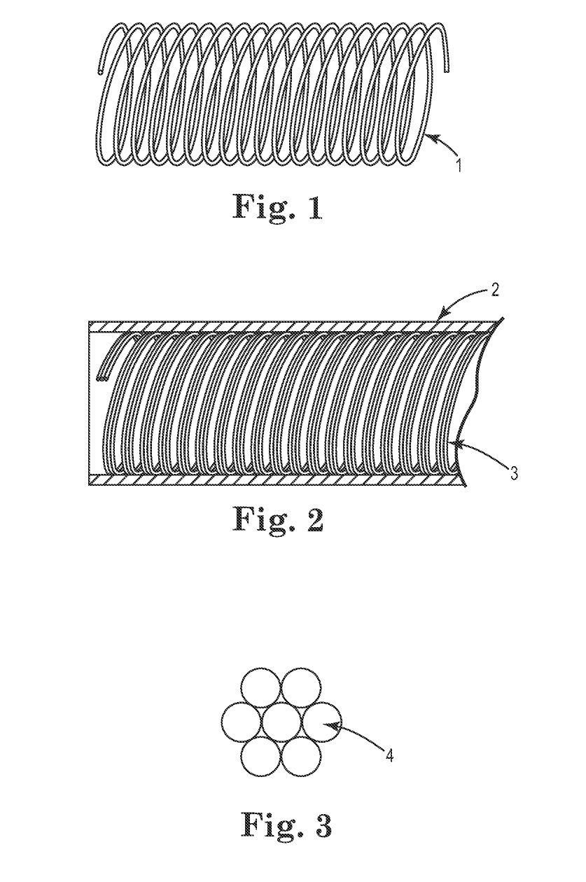

FIG. 1 illustrates a single coil 1. Single coils 1, in accordance with FIG. 1, made of fine-grained stabilized Ta with a specific electric resistance of 14.3 μΩcm, or fine-grained stabilized Nb with a specific electric resistance of 16 μΩcm, are realizable with a mechanical strength of more than 1000 MPa. Therefore, with regard to mechanical strength, said coils 1 are superior to coils made of more conductive metal, for example, gold, silver, copper, and aluminum. Furthermore, the coils 1, according to one embodiment, are superior to mechanically stronger metal with regard to their electrical conductivity. In one embodiment, this contrast especially strongly with the few biocompatible metals, such as MP35N®. The coils, according to one embodiment, can be insulated optionally with plastic, for example, silicone, polyurethane, PTFE, or ETFE, around the conductor or the outer coil diameter.

Multicoils, according to one embodiment, can exhibit wires, which are insulated against each other, thereby connecting different electrical contacts with each other. The multicoil is positioned around its radius in an insulation, for example, a silicone tube. FIG. 2 illustrates a cable consisting of a multicoil 3 with three wires in an electrical insulation 2. Optionally, the individual wires can be insulated against each other with plastic. Thereto, strands made of TaNb10W7.5, which exhibit a mechanical strength of more than 1200 MPa at an electric resistance of 18.8 μΩcm, are suitable.

According to FIG. 3, seven strands 4 are twisted to a stranded wire. With strands made of fine-grained stabilized Ta or Nb, these strands 4, with regard to mechanical strength, are far superior to coils made of more conductive metal, and superior to mechanically stronger metal with regard to their electrical conductivity. In one embodiment, this contrasts especially strongly with the few biocompatible metals, such as MP35N®.

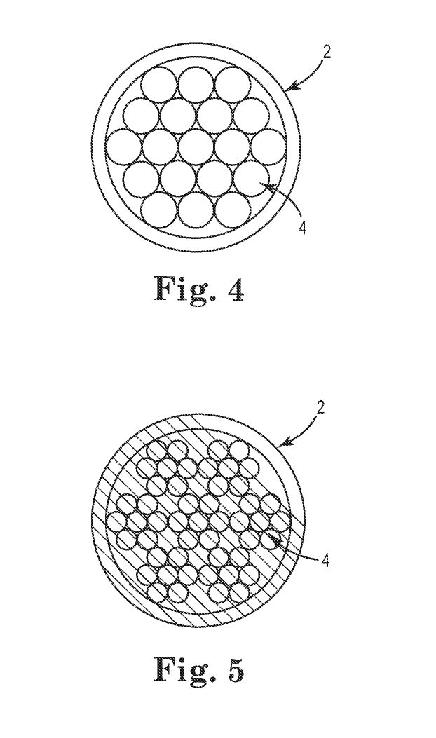

According to FIG. 4, nineteen strands 4 are twisted to a stranded wire within the insulation 2. Thereto, strands made of TaNbWZr, for example, are suited.

In FIG. 5, seven, possibly insulated, stranded wires, within an optional outer insulation 2, are twisted together in accordance with FIG. 3. Thereto, stranded wires made of O-doped TaNbW, for example, are suited.

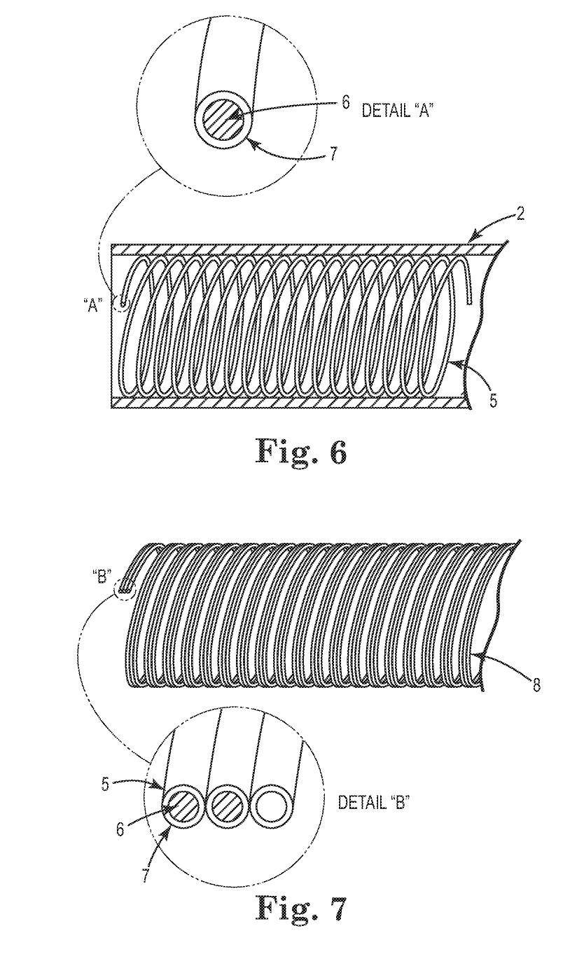

In FIG. 6, a clad material 5 with a core 6 and a jacket 7 is arranged, analogous to FIG. 1, in an optional electrical insulation, for example, a silicone tube. The core 6 consists of a metal with better conductivity than the jacket, for example, made of Ag. The jacket consists of a metal based on Nb or Ta with greater mechanical strength than the core. Fine-grained stabilized Ta or Nb is suited for jackets with cores made of Ag but also as core inside jackets made of Ta or Nb alloys with greater mechanical strength. A clad material made of fine-grained stabilized Ta or Nb and filled with 33% Ag, relative to the profile, exhibits a specific electric resistance of approximately 4 μΩcm. Compared to a clad material made of MP35N® which is filled with 41% Ag and which also exhibits a specific electric resistance of approximately 4 μΩcm, a significantly greater jacket thickness can be achieved for the jacket through the use of fine-grained stabilized Ta, which contributes to the strength of the clad material and is less susceptible to material defects. In addition, precious metal is saved.

In FIG. 7, a clad material compound 8 made of three clad materials 5, each consisting of a core 6 and a jacket 7, is illustrated. As in FIG. 6, the core 6 consists of the metal with better conductivity, for example, Ag. The jacket consists of the metal with great mechanical strength on Nb or Ta basis, for example, TaNbZr.

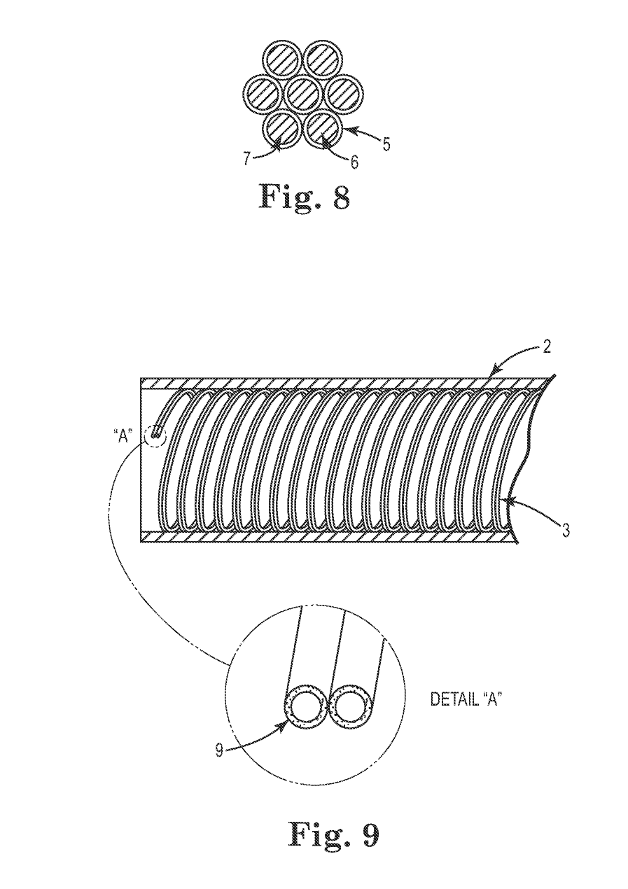

The cable in accordance with FIG. 8, compared to FIG. 3, contains seven clad materials 5, each with a core 6, for example, a silver core, and a jacket 7 made, for example, from TaNbZr.

According to FIG. 9, a multicoil, for example, made of Ta or Nb wire, surface-doped with O, is arranged within an electrical insulation, for example, wrapped with a silicone tube.

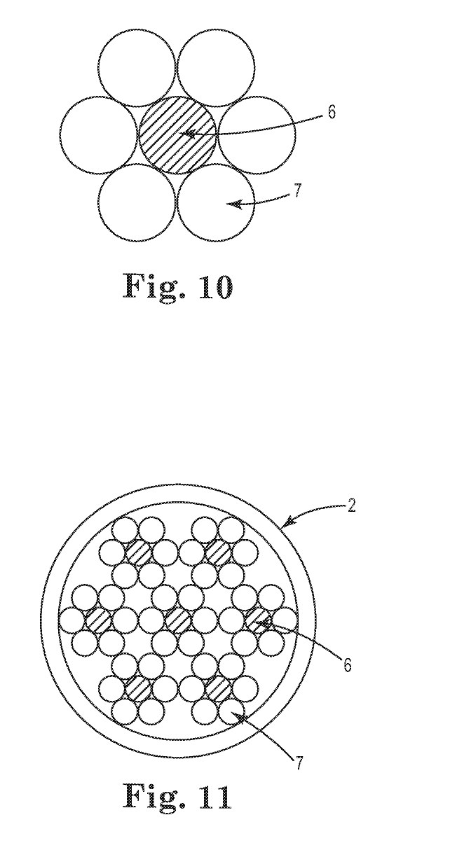

FIG. 10 differs from FIG. 3 in such a way that the stranded wire possesses a centrally positioned conductor 6 with high conductivity, for example, an Ag wire, which was wrapped with 6 wires on Ta or Nb basis, for example, fine-grained stabilized tantalum. In one embodiment, for example, fine-grained stabilized tantalum or niobium wires can be used as centrally positioned conductor, which consist of 6 wires made on the basis of Ta or Nb alloys.

In FIG. 11, seven units, in accordance with FIG. 10, are arranged within an insulation 2. Therefore, this stranded wire consists of 7 1×7 strands, wherein, for example, each Ag wire was wrapped with 6 TaNbW wires. The wires wrapped around the conductor 6 with high conductivity, for example, silver wire, are arranged elastically within the coil as jacket around a core, for example, a silver core, and provide the wire with excellent long-term stability for medical applications where the wire must withstand movements over a long period of time. Biocompatible alloys improve the dependability of the cable, and even damage to the cables will not be detrimental for the patient. In this embodiment, in accordance with FIG. 10, seven cables are arranged into one cable which keeps the inner cables in place with an additional outer insulation.

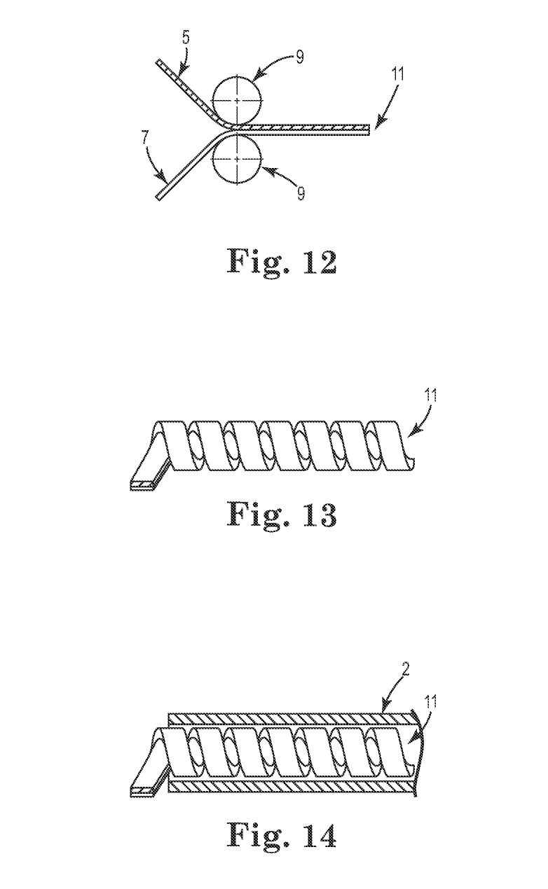

For the manufacture of a laminate in accordance with FIG. 12, ribbons of varying metals are laminated to a ribbon by means of roll cladding. The roll cladding of only two ribbons impresses with the simplicity of the method. Roll cladding with three ribbons allows for sandwich structures. The roll cladding causes cold welding between the materials, whereby the varying metals adhere to each other. In order to further improve the contact between the materials, additional heat can be applied. This supports the diffusion of the atoms of the materials involved. In one embodiment, the ribbon is coated after roll cladding with an elastic, electrically insulating, biocompatible plastic. Thereby, the biocompatibility is improved. In accordance with FIG. 13, the ribbon is formed into a coil, which is fused with plastic, for example, into a strand, in accordance with FIG. 14. In one embodiment, it has proven successful to coil the laminate onto a rod and to coat the coil thereupon with plastic, in accordance with FIG. 14.

Finally, the insulated ribbon is connected on one end to the electrode and on the other end to a plug for the cardiac pacemaker.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. A stranded wire comprising:
a plurality of coils;
wherein the stranded wire is configured as an electrical connection between an electrical stimulation device that is connected to the proximal end of the stranded wire, and an electrode connected to the distal end of the stranded wire; and
wherein at least one coil comprises a tantalum or niobium-based metal.

2. The stranded wire of claim 1, wherein the tantalum or niobium-based metal exhibits a strength of more than 1000 MPa and a specific electric resistance of less than 100 μΩcm.

3. The stranded wire of claim 1, wherein the niobium or tantalum-based metal is doped with at least one element from a group comprising P, B, O, C, N, Si, F, Y, La, Ce, Pr, Nd, Sm, Eu, Gd, Tb, Dy, Ho, Er, Tm, Yb, and Lu, and that exhibits a specific electric resistance <20 μΩcm.

4. The stranded wire of claim 1, wherein the niobium or tantalum-based metal is a gradient material, surface-doped with at least one element from a group comprising P, B, O, C, N, Si, F, Y, La, Ce, Pr, Nd, Sm, Eu, Gd, Tb, Dy, Ho, Er, Tm, Yb, and Lu.

5. The stranded wire of claim 1, wherein the niobium or tantalum-based metal is alloyed with at least one other element from a group comprising niobium, tantalum, tungsten, zirconium, and molybdenum, and exhibits a strength of more than 1200 MPa.

6. The stranded wire of claim 1, wherein at least one wire of the stranded wire comprises a metal with electrical conductivity of less than 12 μΩcm, and that the metal with better conductivity is surrounded by a body made of tantalum or niobium-based metal.

7. The stranded wire of claim 6, wherein the tantalum or niobium-based metal is doped with one element from a group comprising P, B, O, C, N, Si, F, Y, La, Ce, Pr, Nd, Sm, Eu, Gd, Tb, Dy, Ho, Er, Tm, Yb, and Lu.

8. The stranded wire of claim 6, wherein the metal with the higher electrical conductivity is embedded in a body made of a niobium or tantalum alloy, which contains at least one other element from a group comprising niobium, tantalum, tungsten, zirconium, and molybdenum.

9. A helix coil configured as an electrical connection between an electrical stimulation device that is connected to the proximal end of the coil, and an electrode that is connected to the distal end of the coil, wherein the coil comprises a tantalum or niobium-based metal.

10. The helix coil of claim 9, wherein the tantalum or niobium-based metal is doped with one element from a group comprising P, B, O, C, N, Si, F, Y, La, Ce, Pr, Nd, Sm, Eu, Gd, Tb, Dy, Ho, Er, Tm, Yb, and Lu, and exhibits a specific electric resistance <20 μΩcm.

11. The helix coil of claim 9, wherein the tantalum or niobium-based metal is a gradient material, surface-treated with at least one element from a group comprising P, B, O, C, N, Si, F, Zr, Y, La, Ce, Pr, Nd, Sm, Eu, Gd, Tb, Dy, Ho, Er, Tm, Yb, and Lu.

12. The helix coil of claim 9, wherein the tantalum or niobium-based metal is alloyed with at least one other element from a group comprising niobium, tantalum, tungsten, zirconium, and molybdenum, and exhibits a strength of more than 1200 MPa.

13. The helix coil of claim 9, wherein the coil exhibits at least one metal with electrical conductivity of less than 12 μΩcm, and that the metal with better conductivity is surrounded by a body made of tantalum or niobium-based metal.

14. The helix coil of claim 13, wherein the tantalum or niobium-based metal is doped with one element from a group comprising P, B, O, C, N, Si, F, Y, La, Ce, Pr, Nd, Sm, Eu, Gd, Tb, Dy, Ho, Er, Tm, Yb, and Lu.

15. The helix coil of claim 13, wherein the tantalum or niobium-based metal is alloyed with at least one other element from a group comprising niobium, tantalum, tungsten, zirconium, and molybdenum, and exhibits a strength of more than 1200 MPa.

16. A metallic composite comprising:
an outside portion comprising a tantalum or niobium-based metal;
wherein the metallic composite is configured as an electrical connection between an electrical stimulation device and an electrode; and
wherein the electrical stimulation device is coupled to the proximal end of the metallic composite and the electrode is coupled to the distal end of the metallic composite.

17. The metallic composite of claim 16, wherein the tantalum or niobium-based metal exhibits a strength of more than 1000 MPa and a specific electric resistance <100 μΩcm.

18. The metallic composite of claim 16, wherein the metallic composite is coiled.

19. The metallic composite of claim 16, wherein the tantalum or niobium-based metal is doped with one element from a group comprising P, B, O, C, N, Si, F, Y, La, Ce, Pr, Nd, Sm, Eu, Gd, Tb, Dy, Ho, Er, Tm, Yb, and Lu.

20. The metallic composite of claim 16, wherein the tantalum or niobium-based metal is alloyed with at least one other element from a group comprising niobium, tantalum, tungsten, zirconium, and molybdenum.

21. The metallic composite of claim 16, wherein the electrical stimulation device is a cardiac pacemaker or an implantable defibrillator.

22. The metallic composite of claim 16, wherein the metallic composite is configured as a cable.

23. The metallic composite of claim 16, wherein the metallic composite is configured as an implantable stimulation lead.

24. The metallic composite of claim 23, further comprising a plug for a plug-in connection at the proximal end of the stimulation lead.

25. The metallic composite of claim 16, wherein the metallic composite is applied as a neurostimulator or a peripheral muscle stimulator or a cardial stimulator.
